(19) United States
(12) Patent Application Publication
(10) Pub. No.: US 2002/0055731 A1
(43) Pub. Date: May 9, 2002

(54) METHODS FOR PROMOTING CELL TRANSFECTION IN VIVO

(76) Inventors: ANTHONY ATALA, WESTON, MA (US); MARCELLE MACHLUF, BROOKLINE, MA (US)

Correspondence Address: LAHIVE & COCKFIELD, 28 STATE STREET, BOSTON, MA, 02109, US

(21) Appl. No.: 09/178,373

(22) Filed: Oct. 23, 1998

Related U.S. Application Data
(60) Provisional application No. 60/062,810, filed on Oct. 24, 1997.

(51) Int. Cl. A61M 31/00

(52) U.S. Cl. 604/522; 604/501; 604/20

(57) ABSTRACT

Methods, systems, and devices for promoting ultrasound-mediated gene transfection in vivo are disclosed. The methods and devices of the invention administer a gene formulation to the subject; and apply ultrasound energy to the subject, such that cell transfection is promoted. The ultrasound energy can be provided by an external source or an ultrasound source disposed inside the subject's body. For example, the source of ultrasound energy can be disposed on a catheter. The compositions suitable for ultrasound-mediated transfection can include genes (and plasmids containing such genes) in a pharmaceutically acceptable carrier and/or liposomes suitable for injection into a target site, as well as implantable degradable matrix carriers.

### REFERENCE TO RELATED APPLICATION

The present application claims the benefit of, and incorporates by reference, the commonly owned co-pending U.S. provisional application No. 60/062,810, filed Oct. 24, 1997.

### BACKGROUND OF THE INVENTION

Gene therapy, the genetic modification of a cell or organism with a gene or genes, has great promise as a method for treating a wide variety of conditions, including genetic conditions resulting from genetic defects or acquired conditions such as cancer. To realize the promise of gene therapy, safe and effective methods for introducing a gene (and its control sequence) into cells of an organism must be developed. Such methods should be capable of introducing a gene into a cell in such a way as to permit the gene product to be expressed in the cell, without destroying the cell. In many cases it would be useful for the introduced gene to become stably integrated into the genome of the cell or organism. Stable integration assures that the gene product will continue to be produced over an extended period, and also permits the gene to be transmitted to daughter cells of the original host cell.

Over the past decade, different techniques for gene delivery to mammalian cells have been extensively studied. Introduction of the gene into cell can take place ex vivo by transfecting cultured cells and then transplanting them into the recipient, or the gene can be transferred directly into cells, tissues and organs in vivo. According to conventional methods, both transfection strategies are carried out using the aid of viral or non-viral vectors which enhance the delivery of genes into an intracellular compartment and/or nucleus, where they are expressed. Most gene therapies have involved the use of viral-based vectors such as retrovirus and adenovirus, as carriers of genes in vitro and in vivo. However, retrovirus, in vivo, can integrate into the cellular genome and may inactivate host tumor suppression gene or activate proto-oncogenes. Also, retrovirus can promote gene transfection of only limited size genes (7 kB) and can infect only dividing cells, whereas much mammalian tissue consists primarily of non-dividing cells. Adenovirus, the second most commonly used viral vector, has the disadvantage of infecting all tissue, including germ cells, when delivered in vivo. Moreover, adenoviruses are immunogenic and non-integrating, rendering it unsuitable for long term expression. To circumvent these problems, non viral techniques such as direct injection of naked DNA, liposomes and lipofection, electroporation or polymers and other chemical vectors have been used. However, although these methods are generally deemed safe, they have met with limited success and low transfection capability.

Ultrasound frequencies ranging between 1-3 MHz have been used for certain therapeutic applications, such as for local pain relief, or treatment of musculoskeletal injuries or chronic inflammatory conditions. Ultrasound is also used for increasing the permeability of skin to small molecule drugs and proteins (a process known as sonophoresis). The permeability of hydrocortisone, lidocaine, salicylic acid and other therapeutic agents have been shown to be increased due to the application of 1-3 MHz frequencies and intensities ranging between 0.2 to 2 w/cm². Sonication and ultrasound have been used in a limted manner for plasmid delivery in vitro but not in vivo. To date, however, applications of ultrasound transfection have been few and have met with limited success.

### SUMMARY OF THE INVENTION

The present invention relates to methods and systems for promoting the transfection of cells, including mammalian cells, with genes, in vitro or in vivo. Thus, the methods and systems of the invention are useful, e.g., for gene therapy or other applications in which expression of a gene in a host cell is desirable. The invention provides a novel technique for gene transfection of cells in vitro and organs in vivo, including non-dividing and dividing cells. Accordingly, the methods and systems of the invention will have a variety of uses.

In one aspect, the invention provides a method for promoting cell transfection in a subject. The method includes the steps of administering a gene formulation to the subject; and applying ultrasound energy to the subject, such that cell transfection is promoted in the subject. The ultrasound energy can be provided by an ultrasound source disposed inside the subject's body. For example, the source of ultrasound energy can be disposed on a catheter. The step of administering a gene formulation to the subject can include administering one or more genes formulated in liposomes.

In another aspect, the invention provides a system for promoting cell transfection in a subject. The apparatus includes means for administering a gene formulation to the subject; and means for applying ultrasound energy to the subject. The means for applying ultrasound energy to the subject can be an ultrasound transducer mounted on a catheter, or a needle operatively connected to an ultrasound transducer.

In another aspect, the invention provides a catheter for promoting cell transfection in a subject. The catheter includes an elongate tubular assembly having at least one lumen and an opening at or near the distal end of the catheter; the distal end of the lumen being in fluid communication with the opening; and a source of ultrasound energy disposed at or near the distal end of the catheter. The proximal end of the catheter can be provided with an adapter for introduction of a fluid into the lumen. In certain embodiments, the catheter further includes a reservoir in fluid communication with to the adapter.

### BRIEF DESCRIPTION OF THE DRAWINGS

FIG. 1 is a bar chart showing the results of ultrasound-mediated gene transfer in mammalian fibroblast cells in vitro;

FIG. 2 is a bar chart showing the results of ultrasound-mediated gene transfer in mammalian urothelial cells in vitro;

FIG. 3 is a bar chart showing transfection of ultrasound-exposed, fibroblast cells in vitro by electroporation;

FIG. 4 is a bar chart depicting pGL3-Luc in vivo gene transfer into bladder cells promoted by ultrasound;.

FIG. 5 is a bar chart depicting pVEGF-AP in vivo gene transfer into skin cells promoted by ultrasound;.

FIG. 6 is a schematic illustration of a catheter delivery device for ultrasoundmediated gene transfer according to the invention; and

FIG. 7 is a schematic illustration of an alternative device for ultrasound-mediated gene transfer according to the invention.

### DETAILED DESCRIPTION OF THE INVENTION

The present invention provides systems, methods and compositions for promoting gene transfection in vivo or in vitro. The systems or devices, methods, and compositions of the invention can be used for, e.g., gene therapy of a subject. As used herein, the term “subject” or “patient” refers to an animal into which a gene is to be introduced. Subjects include higher organisms including warm-blooded animals such as birds and, more preferably, mammals, including cats, dogs, rats, mice, sheep, goats, cattle, horses, pigs, non-human primates, and, in certain preferred embodiments, humans. However, it will be appreciated that the invention also permits ultrasound-promoted transfection of cells of lower organisms such as plants.

The term “gene” as used herein, refers to a nucleic acid (e.g., DNA or RNA) or nucleic acid construct or expression vector (such as a plasmid) which encodes a gene product such as a protein or protein fragment. Unless otherwise indicated, the term “gene” also is intended to include any control or promoter regions necessary for expression of the gene product in a cell to be transfected. Thus, a solution of a gene can include a solution of a nucleic acid which comprises a gene sequence which encodes a gene product, such as a protein, as well as an upstream promoter sequence or regulatory region which promotes expression of the gene sequence in the host cell. “Expression vector” refers to a replicable DNA construct used to express DNA which encodes the desired protein and which includes a transcriptional unit comprising an assembly of (1) genetic element(s) having a regulatory role in gene expression, for example, promoters, operators, or enhancers, operatively linked to (2) a DNA sequence encoding a desired protein which is transcribed into MRNA and translated into protein, and (3) appropriate transcription and translation initiation and termination sequences (which may vary according to the host cell). Genes, nucleic acids, and expression vectors can be provided and purified according to standard methods of molecular biology.

The protein or protein fragment which is encoded by a gene can be a naturally-occurring protein or portion thereof, or the protein can be a synthetic construct, e.g., a mutant protein or a fusion protein. A gene can be a gene which naturally occurs in the subject (e.g., to increase expression of a gene product which is produced in insufficient amounts in the subject's body), or the gene can be a foreign or exogenous gene, e.g., a gene which is not found naturally in the subject's body. A foreign gene can include a gene which is naturally found in an organism other than the subject, or a foreign gene can be a synthetic gene construct.

As used herein, the term “transfection” means the introduction of a heterologous nucleic acid, e.g., an expression vector, into a recipient cell by nucleic acid-mediated gene transfer.

For use in gene therapy, cells can be transfected in vitro, followed by introduction of the transfected cells into the body of a subject. Alternatively, cells can be transfected in vivo. The in vivo transfection procedure is potentially a simple, one-step procedure, rather than the two-step in vitro procedure. In a preferred practice of the methods of the invention, a gene is introduced (e.g., as a solution, suspension, emulsion, or the like, as described below) directly into the body of the subject, followed by application of ultrasound energy to promote cell transfection.

In one aspect, the invention provides systems and devices for promoting cell transfection in vivo, e.g., in the body of a subject. The system includes means for introducing a gene into the body of the subject, and means for applying ultrasound energy to (at least a portion of) the body of the subject. The means for applying ultrasound energy is preferably structurally or functionally linked to the means for introducing the gene into the body of the subject.

A means for introducing a gene into the body of the subject can be provided according to a variety of methods, some of which are known in the art. For example, a solution or formulation of a gene (i.e., a nucleic acid) can be provided by dissolving or suspending a gene in a pharmaceutically acceptable carrier. Gene formulations include solutions, suspensions, emulsions, liposomes, and the like. A gene formulation can include more than one gene in the formulation, for simultaneous transfection of cells with two or more genes. It will be appreciated that a gene formulation can be a solution in an aqueous solvent, but, in certain embodiments, a solution or suspension in a nonaqueous fluid, such a perfluorocarbon, is preferred. For example, when it is desired to transfect cells in the lung of the subject, it may be desirable to avoid filling the lung with an aqueous solution. Thus, the lung can be partially or wholly filled with a solution of a gene in an oxygenated perfluorocarbon carrier, thereby providing adequate oxygenation of lung tissues while providing a gene formulation to the lung tissues. For examples of perfluorocarbons suitable for oxygenation of lung tissues with simultaneous application of ultrasound to the lung, see, e.g., U.S. Pat. No. 5,562,608 to Sekins et al.

The solution or formulation containing the gene can then be delivered into the body of the subject by various means, e.g., by injection (e.g., subcutaneous, intramuscular, intraperitoneal, and the like), instillation, cannulation, implantation and the like. Thus, means for introducing the gene into the subject's body include needles, catheters, biodegradable implants, and the like. A gene formulation can be administered systemically, e.g., by intravenous injection of the gene formulation into the circulation. Alternatively, the gene can be administered to confine the gene formulation to a particular target tissue or area. For example, the gene formulation can be introduced by catheter (see, e.g., U.S. Pat. No. 5,328,470) into a hollow organ (such as bladder, prostate, lung, uterus, and the like) or body cavity (e.g., peritoneal cavity, skull, and the like), or by stereotactic injection (e.g. Chen et al. (1994) PNAS 91: 3054-3057). Suitable catheters for the introduction of therapeutic agents (i.e., drugs) or diagnostic agents (e.g., dyes) into a variety of organs or body cavities are known, and can be adapted for use in the present invention. In a preferred embodiment, the means for introducing a gene into the body of the subject is a catheter having a catheter lumen which, when introduced into the patient's body, fluidly communicates a source of a gene formulation, such as a reservoir of a gene solution, to a hollow organ or body cavity.

A nucleic acid can also be suspended in a carrier such as, e.g., an ointment or a lotion, for topical application to an exposed organ or tissue of the subject, such as skin, eyes, or mucous membranes. Similarly, a transdermal patch can be employed for administration of a nucleic acid to skin. Thus, in certain embodiments, the means for introducing a gene into the body of the subject can be a transdermal patch. In the case of application to the skin, increased transport of the gene to skin cells, or to tissues underlying the skin, can be provided by use of an additive such as a polyethylene glycol (PEG), which are known for increasing permeability of skin to pharmaceutical agents. Conveniently, for topical application a gene formulation can be provided in a lubricant base, which permits the use of a conventional ultrasound probe (e.g., an imaging ultrasound apparatus) to provide ultrasound energy for promoting transfection, without need for an additional conventional lubricant such as is generally used with conventional probes.

Furthermore, the nucleic acid can be suspended in or admixed with a suitable solid or semisolid carrier, e.g., a biodegradable carrier, for administration to the subject by implantation. The pharmaceutical preparation can consist essentially of the gene delivery system in an acceptable diluent, or can comprise a slow release matrix in which the gene delivery vehicle is imbedded. Various slow release polymeric devices have been developed and tested in vivo in recent years for the controlled delivery of drugs. A variety of biocompatible polymers (including hydrogels), including both biodegradable and non-degradable polymers, can be used to form an implant for the sustained release of a gene. However the gene or nucleic acid is administered to the patient, the gene or nucleic acid is administered in a form which permits the nucleic acid to contact cells of the tissue(s) or organ(s) which are to be transfected.

A means for applying ultrasound energy to the body of the subject can be any of a variety of devices for administering ultrasound, some of which are known in the art. For example, many devices for clinical imaging or therapeutic treatment using ultrasound are known. Such devices include ultrasound imaging probes, ultrasound baths, horns, needles, and the like. Such devices can provide ultrasound energy to large or small portions of a subject's body. A source of ultrasound energy can be external to the subject's body (e.g., a handheld ultrasound probe which is applied to the patient's skin and projects ultrasound energy into the body), or internal (e.g., a catheter having an ultrasound transducer located at a distal end of the catheter, or an implantable ultrasound source).

In a preferred embodiment, in a system according to the present invention, the means for applying ultrasound energy is structurally or functionally linked to the means for introducing the gene into the body of the subject. Thus, in one preferred embodiment, the means for introducing the gene is mounted on or connected to the means for applying ultrasound energy; or the means for applying ultrasound energy can be mounted on the means for introducing the gene. For example, in a preferred embodiment, the invention provides a catheter; the catheter comprises an elongate, preferably flexible tubular assembly having at least one lumen extending along the elongate axis. The catheter is configured for introduction into the subject's body, and has an opening at or near the distal end of the catheter; the distal end of the lumen of the catheter is in fluid communication with the opening. The proximal end of the catheter can be provided with an adapter or port for introduction of a fluid into the lumen. The catheter can also include a source of ultrasound energy (e.g., a transducer) disposed at or near the distal end of the catheter. In this embodiment, the catheter is adapted to provide a gene formulation, such as a solution, from a fluid source (e.g., an reservoir) which can be external to the patient's body, through the opening in the catheter tip, and to provide localized ultrasound energy to the subject's body at or near the location to which the gene formulation is introduced. For example, a catheter of the invention can be adapted for placement in the bladder of the subject to fill the bladder with a solution of a gene (see, e.g., Example 3). Ultrasound energy can then be selectively applied to the fluid-filled bladder by the source of ultrasound disposed at the catheter tip. A catheter of the invention can be provided with additional lumens, e.g., a lumen for draining fluids from a site in vivo, and if appropriate, with suitable drainage openings(s) and ports (at the proximal end of the catheter) for connection to drainage bags or other means for draining fluids.

Ultrasound transducers suitable for placement at a distal end of a catheter are known and can be used in the devices of the present invention. For example, U.S. Pat. No. 5,676,151 to Yock discloses a catheter having an ultrasound transducer disposed at the distal catheter end. The ultrasound source (e.g., the transducer) can be constructed to provide an omnidirectional, or, alternatively, directional source of ultrasound energy. In certain embodiments, where it is advantageous to provide ultrasound energy to most or all of the tissue which surrounds the catheter tip, the ultrasound source can be omnidirectional, e.g., by use of a single omnidirectional transducer, or multiple transducers arranged to provide ultrasound in all directions. A directional ultrasound transducer can be rotated, e.g., by rotating the catheter, to provide ultrasound in all directions around the catheter. In this embodiment, the catheter can be provided with means for rotating the catheter, e.g., a torque cable extending through the catheter and adapted for connection to an external motor, e.g., as described in U.S. Pat. No. 5,676,151 to Yock.

Alternatively, if selective application of ultrasound to tissue is desired, a shield can be placed proximal to the ultrasound source to block ultrasound energy in a proximal direction. Similarly, a shield can be positioned to direct ultrasound energy from the transducer in a selected direction from the catheter body; for example, to provide ultrasound energy to tissue on one side of the catheter while substantially blocking the transmission of ultrasound energy to tissue on an opposed side of the catheter, thereby providing differential ultrasound irradiation to tissue on opposed sides of the catheter.

The ultrasound source is preferably connected to a power supply and to suitable control means for regulating the ultrasound signal produced by the ultrasound source. The power supply and control means (e.g., circuitry) can be disposed external to the body, e.g., at a proximal end of the catheter, and be operatively connected to the ultrasound source, e.g., by means of wires running from the ultrasound source, through a conduit in the catheter, to the power supply and control means. The control means can be provided with a user interface for setting parameters such as the power, duty cycle, and pulse duration of the ultrasound energy provided by the ultrasound source. Suitable control means will apparent to one of ordinary skill in the art. It will be appreciated that an ultrasound transducer such as a piezoelectric crystal can be operated as a receiver for ultrasound waves reflected from tissue surrounding the transducer. Thus, the control means can be provided with appropriate receiver circuitry to provide imaging capability to the catheter of the invention. Such imaging capability is useful for accurately positioning the catheter of the invention adjacent a target tissue or organ.

Suitable perfusion lumens and ports may be provided in such a catheter to provide for the administration of gene formulations to a target site in vivo. Selection of a port or adapter (e.g., a Luer adapter) will be routine for the skilled artisan.

In certain embodiments, the catheter further includes a sensor or sensors, such as a pressure sensor (e.g., a solid-state piezoresistive diaphragm-based sensor) or a temperature sensor (e.g., a thermistor), operatively connected (e.g., via lead wires) to appropriate sensor circuitry, for monitoring the tissue while therapy is provided. A pressure sensor can be desirable where, for example, a catheter of the invention is placed in a hollow organ such as the bladder, to avoid overfilling the organ and causing discomfort or damage to the patient. Ultrasound energy can heat surrounding tissue; thus, in certain preferred embodiments, a catheter of the invention includes a temperature sensor, to ensure that tissue is not overheated and damaged by the application of ultrasound. Of course, in certain embodiments, heat can be applied as an adjunctive therapy, e.g., to destroy cancerous cells. Thus, for example, a catheter of the invention can be positioned within or adjacent a cancerous mass to utilize dual modes of cancer therapy, e.g., gene therapy, as described herein, in combination with heat therapy to destroy cancer cells.

In another embodiment, a catheter of the invention can comprise an elongate, preferably flexible tubular assembly having at least one lumen, configured for introduction into the subject's body, with an opening at or near the distal end of the catheter as described above and further comprises a receiver for detecting ultrasound energy disposed at the distal end of the catheter. The receiver (which may be also be a transducer) can be operatively linked, e.g., through lead wires and feedback control circuitry, to an external source of ultrasound, to provide selective control of the amount of ultrasound energy which is applied to the target tissue. For example, the catheter can be inserted into the bladder of the patient as described infra, and the bladder filled with a solution of a gene. Ultrasound energy can then be applied to the bladder from an external source, e.g., an external ultrasound probe applied to the skin overlying the bladder. The ultrasound receiver disposed at the catheter tip can detect the ultrasound energy applied to the catheter tip, and to the fluid-filled bladder, and control signals can be sent to the ultrasound source, e.g., through a control circuit or a radio control system, to increase or decrease the amount of ultrasound energy applied to the bladder.

A catheter of the invention can be introduced according to any method known in the art. For example, a catheter can be introduced through the femoral artery to access the heart or vascular system. A catheter can also be placed through the urethra into the bladder, or further into the ureters to access kidney. A catheter can be inserted through the nose to access the brain. Other suitable catheter placements will be apparent to the skilled surgeon.

In another embodiment, the invention provides a hollow needle which is operatively connected to an ultrasound source, e.g., by mounting thereon. The hollow needle can be connected through a lumen to a reservoir of a gene formulation, such as a syringe, to provide means for administering the gene formulation to the patient, e.g., by subcutaneous injection. The needle is also connected to the ultrasound source such that the needle is capable of transmitting ultrasound energy through the needle body (e.g., the needle sidewall) to the tissue of the subject's body. Thus, the needle should be sufficiently strong and rigid to be capable of transmitting an appreciable amount of ultrasound energy to the patient's tissue.

In another embodiment, the invention provides an implantable device for promoting cell transfection. The device comprises an implantable source of ultrasound energy and an implantable source of a gene formulation disposed on or near the implantable source of ultrasound energy. For example, the source of the gene formulation can be an implant comprising a gene admixed with a biodegradable polymer vehicle. The implantable source of ultrasound energy can be any source of ultrasound small enough to conveniently implanted, such as a crystal transducer, within a housing, which preferably is formed from or coated with a non-immunogenic, biocompatible material (of which many are known in the art). In a preferred embodiment, the implantable source of ultrasound energy can include a power source such as a battery, within the housing, and suitable control means for regulating the ultrasound signal produced by the ultrasound source. In other embodiments, the implantable source of ultrasound energy can be powered by induction, e.g., by transmission of radiofrequency (RF) energy from an external RF transmitter to an RF receiver within the implant. The external RF transmitter can include appropriate control circuitry for controlling the ultrasound produced by the implantable ultrasound source. For an example of a suitable RF transmitter and receiver arrangement, see, e.g., U.S. Pat. No. 5,094,242. In a preferred embodiment, the source of the gene formulation is an implant (e.g., a polymeric implant) which gradually degrades when ultrasound energy is applied. In this embodiment, application of ultrasound can cause degradation of the polymeric implant, thereby releasing the gene, while simultaneously promoting transfection of cells in the surrounding tissue with the gene thereby released.

Where a gene is introduced systemically, e.g. by intravenous injection, ultrasound-promoted cell transfection can potentially occur in several organs or tissues at once, by administration of ultrasound energy to more than one tissue. For example, after systemic administration of a gene formulation, systemic administration of ultrasound energy (e.g., by partial or total immersion of the subject's body in an ultrasonicating bath) can produce cell transfection in more than one organ simultaneously.

Alternatively, specific transduction in selected target cells can occur by selective application of ultrasound to the targeted tissue. For example, ultrasound energy can be selectively applied, e.g., by application of ultrasound to a specific portion of the subject's body. A variety of devices are known for focused or selective application of ultrasound, including phased arrays of ultrasound transducers. The skilled artisan will appreciate that ultrasound energy can be selectively targeted to almost any organ by an appropriate choice of ultrasound transducers (including arrays), and selection of an appropriate power level.

The source of ultrasound should be capable of providing frequencies, energies, and duty cycles of ultrasound energy suitable for promoting transfection of cells in vivo. Thus, for example, in preferred embodiments, a source of ultrasound is capable of generating ultrasound energy in the frequency range from about 20 kHz to about 3 MHz, more preferably from about 20 kHz to about 1 MHz. The power of the ultrasound energy must be high enough to promote transfection, but not so high as to cause excessive tissue damage, e.g., by burning the tissue or causing excessive disruption of cells. Therefore, the power of the ultrasound energy applied to the target tissue or organ can be in the range from about 0.05 W/cm² to about 2 W/cm², more preferably about 0.1 W/cm² to about 1 W/cm², and still more preferably from about 0.25 to about 0.5 W/cm². The duty cycle of the ultrasound source should preferably be from about 10% to about 60%, more preferably from about 20% to about 50%.

In certain embodiments, the systems and devices of the invention include a reservoir or other source of the gene formulation. A reservoir for the gene formulation can be secured to the device such that the reservoir will be internal or external to the subject's body when the device is in use. Thus, for example, a catheter can include an external reservoir, such as a syringe, which can be filled with a solution of a gene, and which can be used to controllably introduce the gene solution into the subject's body, i.e., through the catheter lumen.

The present invention also provides methods for transfecting cells with a gene, in vivo or in vitro. The inventive methods are useful in gene therapy, for example, to introduce genes encoding therapeutic proteins into target cells of a subject's body.

In one embodiment, the invention provides a method for promoting transfection of cells in vivo. The method comprises administering to a subject in need thereof a formulation of a gene in a pharmaceutically acceptable carrier, and applying ultrasound energy to at least a portion of the subject's body, thereby promoting transfection of cells in vivo.

The formulation of the gene can be a solution, suspension, emulsion, implant, or the like, as described hereinbelow. Thus, the step of administering a formulation of a gene to a subject can comprise injection, instillation, inhalation, oral administration, topical administration, or administration by other routes, e.g., as described herein. In a preferred embodiment, the formulation of the gene is administered by instillation (e.g., into the bladder or another hollow organ) of the formulation through a catheter, e.g., a catheter as described hereinabove. Application of ultrasound to the hollow organ results in transfection of cells at the interior surface of the hollow organ, e.g., the bladder wall. In other embodiments, the formulation of the gene is administered through a needle by injection, e.g., by subcutaneous, intravenous, intramuscular or intraperitoneal injection. Injection of the gene formulation is useful for providing systemic gene therapy or treatment of solid organs.

The step of applying ultrasound energy to at least a portion of the subject's body can include application of ultrasound energy with a variety of ultrasound sources, many of which are known in the art. For example, ultrasound sources having the power and frequency characteristics described hereinabove are generally suitable for use in the methods of the invention. The ultrasound energy can be applied to the entire body of the subject, e.g., by immersing the subject in an ultrasonicating bath. Alternatively, the ultrasound energy can be applied to a portion of the subject's body by use of targeted or selective ultrasound sources, including the devices described hereinabove, as well as conventional ultrasound probes (e.g., probes conventionally used for diagnostic imaging or application of therapeutic ultrasound energy). In a preferred embodiment, the formulation of the gene is administered systemically to the subject, e.g., by intravenous injection, and ultrasound energy is applied to substantially all of the subject's body, thereby promoting cell transfection throughout the subject's body. In other preferred embodiments, the ultrasound energy is selectively applied to a portion of the subject's body, e.g., a selected tissue or organ, to selectively promote cell transfection in the selected portion. However, in certain preferred embodiments, the ultrasound source is not an ultrasonicating bath. It will be appreciated that adequate contact between the target tissue and the ultrasound source should be provided, to ensure efficient transfer of ultrasound energy from the source to the target site. Thus, in certain embodiments, a gel or other material can be applied, e.g., to skin, to promote efficient transfer of ultrasound energy from the source to the skin.

It has been found that ultrasound applied to a subject's body can promote significant transfection in as little 1 minute. Thus, in preferred methods of the invention, the ultrasound energy can be applied for a period of at least about one minute, more preferably for at least about 2 minutes, 5 minutes, 10 minutes, 20 minutes, 30 minutes, or one hour. It will be appreciated that higher levels of ultrasound energy can be more damaging to tissue than lower energy levels; accordingly, high power levels are often applied for shorter time periods than low power levels. It will also be appreciated that certain tissue types require longer ultrasound exposure to promote transfection than do other tissues. For example, it has been found that transfection of skin cells in vivo requires longer application of ultrasound that does transfection of bladder cells. In preferred embodiments, the ultrasound energy is not applied for a continuous period of more than 30 minutes. However, it will be appreciated that the ultrasound energy can be applied in divided applications or as pulses over a more extended period of time. It will also be appreciated that methods of the invention can be repeatedly applied to achieved extended expression of the gene product. For example, if the introduced gene does not become stably integrated into the genome of a host cell, the expression of the gene will generally decline after the initial transfection is performed. Thus, repeated applications of the gene formulation and/or the ultrasound energy may be required to maintain gene expression at a desired level. For example, a subject can be treated at regular intervals, e.g., weekly, to ensure adequate levels of gene expression. One of ordinary skill in the art will be able to determine parameters useful for promotion of transfection of cells in vivo, and for maintenance of gene expression, in light of the teachings herein using no more than routine experimentation.

In certain preferred embodiments, the methods of the invention can be used to treat conditions, such as cancer, atherosclerosis, heart disease, diabetes, and the like. The methods of the invention can also be used to treat conditions resulting from deficiency of growth factors or cytokines in selected tissues.

For example, a treatment for cancer could include targeting the cancer cells for destruction by a toxin as follows. Transfection of cancer cells with a gene which encodes a receptor (e.g., heparin-binding growth factor (HBGF)) for a toxin (e.g., diphtheria toxin) can be accomplished as described herein, preferably by means which permit controlled cell transfection (e.g., limited to the cancer cells), rather than systemic transfection. For example, injection of the gene into a solid tumor, followed by application of ultrasound, as described herein, can limit the transfection substantially to the cancerous cells. Transfected cancer cells would produce the receptor, and could then be killed by administering to the patient an amount of the toxin (e.g., diphtheria toxin) which is not fatal to the patient, but is lethal to the “targeted cancer cells” which express HBGF. The transfected cancer cells, which have been “targeted”, can thereby be selectively killed. It will be appreciated that many other toxin/receptor combinations can be similarly employed.

Atherosclerosis and heart disease can be treated according to the methods of the invention by administering to a subject a gene formulation in which the gene encodes vascular endothelial growth factor (VEGF; see, e.g., U.S. Pat. No. 5,607,918 to Erikkson et al., and references cited therein). VGEF is known to promote angiogenesis. Thus, the angiogenic action of VEGF may be useful in treating ischemic conditions, e.g., by stimulating the development of collateral circulation in cases of arterial and/or venous obstruction, e.g. myocardial infarcts, ischemic limbs, deep venous thrombosis, and similar conditions. The gene formulation is preferably administered specifically to tissues or organs (e.g., blood vessels or heart) which have become damaged or occluded (e.g., by atherosclerotic plaques). Ultrasound is then applied to the affected tissue or organ, and cells are thereby transfected. The transfected cells produce VGEF, promoting formation of collateral circulation and thereby restoring blood flow. Those of skill in the art will appreciate that genes which encode other growth factors or cytokines can be employed to promote growth of other cells or tissues.

Other treatments can be readily applied to patients in need thereof. For example, treatment of diabetes can be achieved by transfecting cells with a gene which encodes an insulin, e.g., human insulin, thereby increasing insulin secretion in the subject.

The methods of the invention can be applied to a subject once or repeatedly to achieve a desired therapeutic result. For example, in certain embodiments, the gene will become stably integrated into the genome of the host cell. In this case, the host cell will continue to express the gene product over an extended period of time. However, in other embodiments, the gene will not become integrated into the host cell genome, and will therefore generally be only transiently expressed, with the amount of gene expression often decreasing over the course of a few days or weeks. In such cases, the gene therapy methods of the invention can be repeated to maintain effective levels of protein expression in the host cells.

The methods of the invention are also useful for anti-sense therapy, i.e., administration of anti-sense nucleic acids to a target cell. In this embodiment, the nucleic acid need not be expressed in the host cell, nor is the host cell transfected. The methods of the invention can provide an effective method of delivering anti-sense nucleic acids into the cell. When anti-sense therapy is desired, repeated applications of the anti-sense nucleic acid and the ultrasound may be desirable to accomplish long-term therapy.

In preferred embodiments, the methods and devices or systems of the invention can be used to promote transfection of cells in organs or tissues such as skin, muscle, liver, kidney, brain, eye, heart, pancreas, intestine, stomach, spleen, lung, bladder, prostate, ovary, uterus, and the like. The methods and systems of the invention can also be used to promote cell transfection in body cavities, whether naturally occurring (e.g., the peritoneal cavity, joints such as knee, elbow, shoulder, hip, and the like) or surgically created (e.g., a surgical wound site, or a cavity left in an organ by surgical resection of a tumor). In a preferred embodiment, the tissue or organ is other than a joint.

The success of ultrasound or electrical potential-mediated transfection can be monitored by methods well known to the skilled artisan. The presence of the exogenous gene, or mRNA transcripts in target host cells can be monitored by routine methods such as Southern blotting (DNA), Northern blotting (mRNA), polymerase chain reaction (PCR), and other assay methods well known to the skilled artisan. Alternatively, the presence of the protein encoded by the exogenous gene, or a marker protein, can be monitored to directly or indirectly measure gene expression. For example, a therapeutic gene can be provided in a nucleic acid construct which also includes a marker gene (e.g., luciferase, see, e.g., Examples 1-3, infra). The presence or absence of the protein product of the marker gene can then be used as a surrogate for the presence or absence of the gene of interest in the target cell.

A gene (or genes) can be formulated to provide increased uptake by or transfection of cells in vivo. For example, it is known that cells can be transfected with formulations of a gene in a liposome preparation. Thus, enhanced transfection can be achieved according to the invention by formulation of the gene into a liposome formulation, followed by application of ultrasound energy to the liposomal gene formulation after administration of the formulation to the subject. The gene preparation can also be formulated to provide “targeted” delivery of the gene to a specific organ. Certain known nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject-gene by the targeted cell. Exemplary gene delivery systems of this type include liposomal derived systems, poly-lysine conjugates, and artificial viral envelopes.

In an exemplary embodiment, a gene can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075). For example, transfection of neuroglioma cells can be carried out using liposomes tagged with monoclonal antibodies against glioma-associated antigen (Mizuno et al. (1992) Neurol. Med. Chir. 32:873876), followed by application of ultrasound to the neuroglioma cells according to the methods of the invention.

In another illustrative embodiment, the gene formulation comprises an antibody or cell surface ligand which is cross-linked with a gene binding agent such as polylysine (see, for example, PCT publications WO93/04701, WO92/22635, WO92/20316, WO92/19749, and WO92/06180). For example, a gene construct can be used to transfect hepatocytic cells in vivo using a soluble polynucleotide carrier comprising an asialoglycoprotein conjugated to a polycation, e.g. poly-lysine (see U.S. Pat. No. 5,166,320). It will also be appreciated that effective delivery of the subject nucleic acid constructs via endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al. (1993) Science 260-926; Wagner et al. (1992) PNAS 89:7934; and Christiano et al. (1993) PNAS 90:2122).

It will be appreciated that additional tissue specificity can result from specificity of transfection provided by the gene delivery vehicle, cell-type or tissue-type expression due to the transcriptional regulatory sequences controlling expression of the gene, or a combination thereof. In other embodiments, initial delivery of the gene is more limited with introduction into the animal being quite localized.

The present invention contemplates pharmaceutically acceptable compositions which comprise a therapeutically-effective amount of one or more genes, optionally formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically-effective amount” as used herein means that amount of a gene, material, or composition which is effective for producing some desired therapeutic effect upon ultrasound-promoted transfection of cells with the formulation of the gene.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting a gene from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, genes can be provided in formulations as pharmaceuticall-acceptable salts. The term “pharmaceutically-acceptable salts” in this respect, refers to relatively non-toxic salts, including alkali or alkaline earth salts such as the lithium, sodium, potassium, calcium, magnesium, and aluminum salts and the like. Representative organic amines useful for the formation of base addition salts include ammonia, ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a gene with the carrier and, optionally, one or more accessory ingredients. In general, the formulations are prepared by uniformly and intimately bringing into association a gene with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a gene of the present invention as an active ingredient. A compound of the present invention may also be administered as a bolus, electuary or paste.

In solid dosage forms of the invention for oral administration (capsules, tablets, pills, dragees, powders, granules and the like), the gene is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of a gene include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agaragar and tragacanth, and mixtures thereof.

Formulations of the pharmaceutical compositions of the invention for rectal or vaginal administration may be presented as a suppository, which may be prepared by mixing one or more genes with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a gene include powders, sprays, ointments, pastes, creams, lotions, gels, solutions, patches and inhalants. The gene may be mixed under sterile conditions with a pharmaceutically-acceptable carrier, and with any preservatives, buffers, or propellants which may be required.

The ointments, pastes, creams and gels may contain, in addition to a gene, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a gene, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a gene to the body. Such dosage forms can be made by dissolving or dispersing the peptidomimetic in the proper medium. Absorption enhancers can also be used to increase the flux of the gene across the skin, as desribed above. The rate of such flux can be controlled by either providing a rate controlling membrane or dispersing the gene in a polymer matrix or gel.

Ophthalmic formulations, eye ointments, powders, solutions and the like, are also contemplated as being within the scope of this invention.

Pharmaceutical compositions of this invention suitable for parenteral administration comprise one or more genes in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the gene from subcutaneous or intramuscular injection. This may be accomplished by the use of a liquid suspension of crystalline or amorphous material having poor water solubility. The rate of absorption of the gene then depends upon its rate of dissolution which, in turn, may depend upon crystal size and crystalline form. Alternatively, delayed absorption of a parenterally-administered gene form is accomplished by dissolving or suspending the gene in an oil vehicle.

Injectable depot forms are made by forming microencapsule matrices of a gene in biodegradable polymers such as polylactide-polyglycolide. Depending on the ratio of drug to polymer, and the nature of the particular polymer employed, the rate of gene release can be controlled. Examples of other biodegradable polymers include poly(orthoesters) and poly(anhydrides). Depot injectable formulations are also prepared by entrapping the gene in liposomes or microemulsions which are compatible with body tissue.

When genes are administered as pharmaceuticals, to humans and animals, they can be given per se or as a pharmaceutical composition containing, for example, 0.0001 to 99.5% (more preferably, 0.001 to 10%, still more preferably 0.01 to 1%) of the gene in combination with a pharmaceutically acceptable carrier. For example, in certain embodiments, the gene can be present in a solution at a concentration of between about 15 μg/ml and 200 mg/ml. In certain embodiments, the gene is present in a formulation at a concentration of about 200 μg/ml.

The preparations of the present invention may be given orally, parenterally, topically, or rectally. They are of course given by forms suitable for each administration route. For example, they are administered in tablets or capsule form, by injection, inhalation, eye lotion, ointrnent, suppository, etc. administration by injection, infusion or inhalation; topical by lotion or ointment; and rectal by suppositories. Injection, instillation, or topical administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrastemal injection and infusion.

The phrases “systemic administration,” “administered systemically,” “peripheral administration” and “administered peripherally” as used herein mean the administration of a gene formulation other than directly into the central nervous system, such that it enters the patient's system and, thus, is subject to metabolism and other like processes, for example, subcutaneous administration.

The gene formulation may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, a gene, and/or the pharmaceutical compositions of the present invention, are formulated into pharmaceutically-acceptable dosage forms by conventional methods known to those of skill in the art.

Actual dosage levels of the gene in the pharmaceutical compositions of this invention may be varied so as to obtain an amount of the gene which is effective to achieve the desired therapeutic response for a particular patient, composition, and mode of administration, without being toxic to the patient.

The selected dosage level will depend upon a variety of factors including the activity of the particular gene employed, the susceptibility of the cells to transfection with the gene by application of ultrasound, the route of administration, the time of administration, the rate of excretion of the particular gene being employed, the duration of the treatment, other drugs, compounds and/or materials used in combination with the particular gene employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of a gene employed in the pharmaceutical composition at levels lower than that required in order to achieve the desired therapeutic effect and gradually increase the dosage until the desired effect is achieved.

While it is possible for a gene to be administered alone, it is preferable to administer the gene as a pharmaceutical composition.

Recent studies have shown that ultrasound waves, through a process known as cavitation, have the ability to allow transdermal delivery of macromolecules that would ordinarily have no permeability through the skin. Other studies have shown that the outermost layer of the epidermis, the stratum corneum, is altered by low frequency ultrasound in such a way as to allow both intracellular and intercellular routes of transdermal delivery.

Experimental results (reporteddemonstrate that 1 MHz ultrasound can increase cell transfection with pGL3-Luc (a marker gene) in vitro and in vivo. Without wishing to be bound by any theory, it is believed that ultrasound (especially at frequencies below 3 MHz, and more preferably below 1 MHz) can cause cavitation and facilitate gene transfer through cell membranes. The experiments described herein with 1 MHz ultrasound indicate that ultrasound at a frequency of 1 MHz can increase cell transfection, while other experiments, not shown, have suggested that higher frequencies (above 3 MHz) are less effective at promoting cell transfection.

Using ultrasound waves for in vivo gene transfer could offer several advantages. Ultrasound is currently used to image nearly every part of the body; thus ultrasound waves have access to almost any tissue in the body. Thus, in preferred embodiments, the methods and devices of the invention can be used to promote transfection of cells in organs or tissues such as skin, muscle, liver, kidney, brain, heart, pancreas, intestine, stomach, spleen, lung, bladder, prostate, ovary, uterus, and the like.

The invention is further illustrated by the following non-limiting examples. In these experiments, three DNA plasmids were used. pGL3-Luc (Promega Co.) contains a reporter gene encoding firefly luciferase which is expressed by an SV-40 promoter enhancer. pCMV-VEGF, a plasmid encoding for human vascular endothelial cell growth factor (VEGF), was constructed by subcloning the cDNA encoding VEGF₁₆₅ into pRc-CMV expression vectors (Invitrogen). VEGF₁₆₅ was highly expressed under the regulation of the CMV promoter. The plasmid pVEGF-Alkaline Phosphatase (pVEGF-AP) was constructed using the cDNA encoding VEGF₁₆₅ upstream of the cDNA encoding placental alkaline phosphatase in the APtag-1 vector. The cDNA encoding chimeric VEGF₁₆₅-alkaline phosphatase was subcloned into the pRc-CMV expression vector (Invitrogen). The chimeric protein was highly expressed and retained its alkaline phosphatase activity and heparin-binding capability. Plasmids were purified from bacterial cultures, using a standard alkaline lysis technique followed by isopropanol precipitation (Qiagen Ltd., Promega).

In the in vivo experiments, human bladder tissue specimens were obtained and processed immediately after surgical removal according to previously established techniques. Briefly, specimens were washed with phosphate buffered saline (PBS, Sigma) and urothelial cells were gently scrubbed from the mucosal surface under sterile conditions. The cells were placed in serum free keratinocyte medium containing 5 mg/ml epidermal growth factor and 50 μl/ml pituitary extract and incubated in a humidified atmosphere chamber containing 5% carbon dioxide at 37° C. Human foreskin specimens were obtained after circumcision, washed several times in sterile PBS (pH 7.4), divided into 1 to 2 mm segments, and plated on tissue culture dishes. Cells were grown on Dulbeco 's Modified Eagle's Medium (DMEM, HyClone Utah) containing 10% fetal bovine serum.

A therapeutic ultrasound applicator was used for all experiments (Ultra Max, Excel Tech; Ontario, Canada). The coupling quality, and the total energy and temperature delivered to cells and tissues could be monitored at all times.

For the in vitro transfection experiments, Luciferase plasmid DNA (40 μg) was added to primary urothelial and fibroblast mammalian cells cultured in six well plates (1×10⁶ cells/well). The ultrasound apparatus was immersed directly into the cell culture media and the cells were exposed to pulsatile ultrasound (0.25 or 0.5 w/cm²), 4 wells for each experiment, for different lengths of time (0, 2, 5, 10, 15 and 30 minutes). Control cells received DNA only, without the application of ultrasound. The transfected cells were tested for luciferase activity at 2 days post transfection. Cell viability was assessed using trypan blue inclusion and [3-(4,5Dimethylthiazol-2yl)-2-5-diphenyl-tetrazolium bromide] (MTT; Sigma).

Plates containing pGL3-Luc in medium or PBS (10 μg/ml), were exposed to ultrasound using the same intensities and exposure times as in the cell transfection experiments (1.4). Immediately after exposure, samples of pGL3-Luc DNA were precipitated with 0.4 ml 3M NaOH, 8 ml of 100% EtOH and incubated at −20° C. overnight. The samples were spun (1 lk RPM, 30 minutes), washed in EtOH and resuspended in 200 μl of Tris-EDTA buffer. The precipitated DNA samples (4 μl) were electrophorised on 1% agarose gel in the presence of ethidium bromide. Non treated DNA samples, i.e. not exposed to ultrasound, were used as controls.

The effect of ultrasound on the ability of pGL3-Luc to express protein was also evaluated. pGL3-Luc DNA samples which were exposed to ultrasound as described, were mixed with fibroblasts cells in a final ratio of 60 μg DNA/10⁶ cells. The cells were exposed to electrophoresis using 200V for 1 ms, and assayed 48 hr later for pGL3-Luc protein expression. As a control, fibroblasts alone without DNA underwent electrophoresis. Cell viability was measured immediately post transfection using trypan blue.

In vivo studies using ultrasound as a vector for transfection were performed on rat bladders and mice skin, using different plasmid DNA's. For the bladder transfection, female Sprague Dawley rats underwent anesthetic induction by metafane inhalation followed by intramuscular injection of ketamine (45 mg/kg) and xylazine (10 mg/kg). A median laparotomy was performed in all animals and the bladders were exposed. Transurethral catheterization was accomplished with 24 G intravenous catheters and the bladders were drained completely. In the experimental group, 0.5 ml of pGL3-Luc (200 μg/ml) was instilled intravesically, and ultrasound (pulse mode, 0.25 w/cm for 20 minutes or 0.5 w/cm for 10 minutes) was applied to each bladder. After ultrasound application, the transurethral catheters were removed, and the laparotomy incisions were closed in two layers. The control group received a similar treatment but with no ultrasound application. Rats were sacrificed at different time intervals (0, 2, 3, 5 and 6 days) post treatment. The bladders were harvested, washed in sterile PBS, and frozen under liquid nitrogen. The frozen bladders were weighted and processed by adding 500 μl of 1× lysis buffer (Nalgen) for 15 minutes at room temperature. Lysate specimens were centrifuged and the supernatants was assayed for total protein (BIO-RAD protein assay) and luciferase activity.

For the skin transfection experiments, NCR mice, 8-12 weeks old, underwent anesthetic induction by metafane inhalation. Two plasmids, pVEGF-AP and pCMVVEGF were used for the skin transfection experiments. The plasmid solution (100 μg) was applied to the skin through a Franze diffusion donor chamber and allowed to equilibrate for 15 minutes before applying the ultrasound energy (1 MHz, 2 w/cm²) for 20 minutes. The mice were sacrificed at 0, 2, 3, and 4 days post transfection and the skins were harvested. Skin harvested from mice transfected with human VEGF-AP was frozen in liquid nitrogen until alkaline phospatase assays and western blot analysis were performed. Skin from mice transfected with human pCMV-VEGF was assayed immunohystochemically.

In order to asses the safety of ultrasound as a transfection vector for internal soft tissues, rat bladders exposed to ultrasound were analyzed histologically using the Tunnel assay. Rat bladders were exposed to ultrasound energies of 0.25 or 0.5 w/cm² for 20 minutes. The bladders were harvested immediately after and 2 days post treatment. Bladders were washed in PBS, immersed in Ornitin Carbamoyl Transferase (O.C.T), and frozen in liquid nitrogen. Cryostat sections (5 μm) were stained using Hematoxylin and Eosin. Tunnel stains for the presence of apoptotic cells were performed according to established protocols.

Expression of human pCMV-VEGF protein in mice skins after ultrasound application was evaluated immunohistochemically. Skin harvested from mice at different time points was immersed in O.C.T and frozen in liquid nitrogen. 5 μm frozen sections of were incubated with poly-clonal anti-human VEGF antibodies (R & P) which do not cross react with mouse VEGF. Bound primary antibody was detected using the avidin-biotin-immunoperoxidase method.

Skin samples were weighed and grounded under liquid nitrogen. The grounded samples were lysed with 500 μl of lysis buffer (20 mM Tris pH 7.4, 0.2 M NaCl, 1% Triton X-100 and 2 mM EDTA, Sigma) for 15 minutes at room temperature. The samples were centrifuged and the supernatants were retrieved for western blot and alkaline phosphatase analyses. For the alkaline phosphatase assay, 50 μl of each sample was mixed with phosphatase substrate buffer (Sigma) and incubated at 37° C. for 1 to 24 hrs. The samples were analysed using an ELISA reader at a wavelength of 420 nm. For the western blot analyses, skin lysates were incubated with heparin Sepharose for 24 hr at 4° C. The Sepharose was washed with buffer (150 mM NaCl, 0.1 % Triton X-100 and 20 mM Tris) followed by PBS and subjected to SDS-Poly acrylamide gel electrophoresis. Proteins were electrophoretically blotted onto Imobilion P membranes (Millipore Corp). Blots were probed with human anti VEGF antibodies (Santa Cruz Calif.). Detection of antibodies was performed by chemiluminescence using an ECL system (Amersham Corp)

### EXAMPLE 1

### In vitro Gene Transfection:

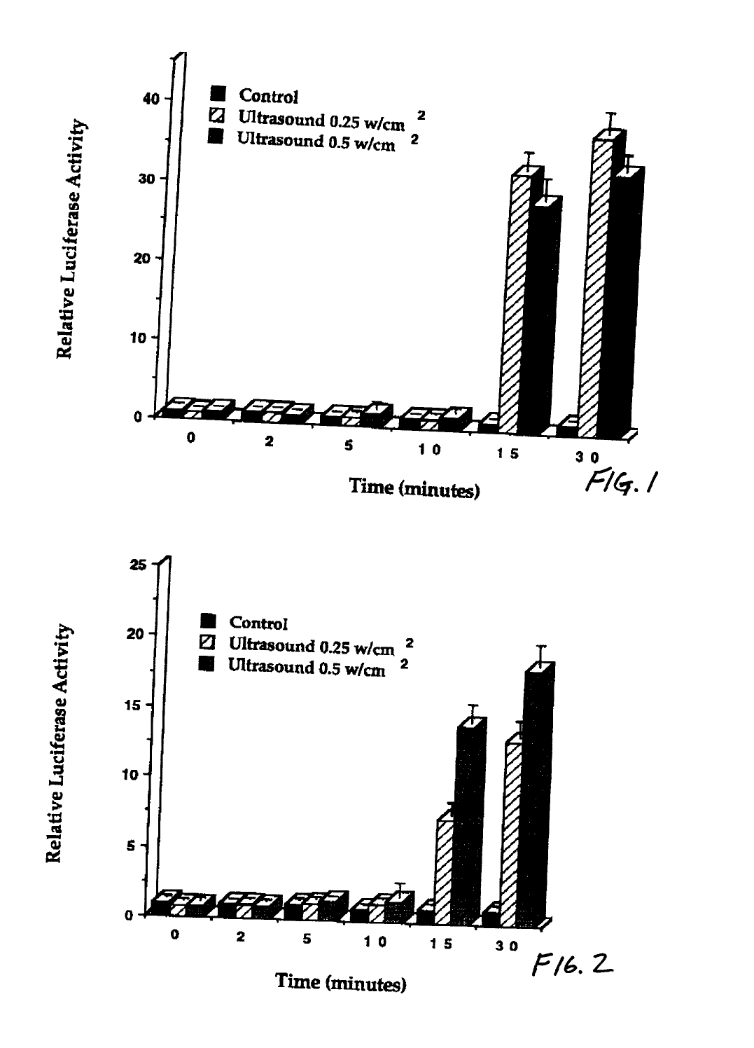

A series of experiments were performed in which the effects of applied ultrasound on in vitro pGL3-Luc transfection of fibroblasts and urothelial cells was assessed. For each ultrasound parameter, 4 wells were assayed, and each experiment was repeated at least twice. FIG. 1 shows the effects of therapeutic ultrasound on pGL3-Luc transfection in mammalian fibroblasts. The cells were exposed to 2, 5, 10, 15 and 30 minutes of ultrasound energy using a pulsed mode (0.25 w/cm²). Fibroblasts were successfully transfected by pGL3-Luc after 15 and 30 minutes of ultrasound application. Luciferase protein expression was 34 and 37 fold higher, respectively, than the control cells which were not treated with ultrasound. Increasing the ultrasound intensity from 0.25 to 0.5 w/cm² gave a similar transfection efficiency. Protein expression was not detected when ultrasound was applied for 2, 5 or 10 minutes.

A similar effect was seen when urothelial cells were treated with pulsed ultrasound (0.25 w/cm², FIG. 2). Ultrasound application for 15 or 30 minutes significantly enhanced pGL3-Luc transfection (7 and 14 fold) in the treated cells compared to the non treated cells. A parameter which significantly affected urothelial cell transfection was ultrasound intensity (FIG. 2). Increasing the intensity from 0.25 to 0.5 w/cm², while applying ultrasound energy for fifteen minutes, elevated cell transfection and protein expression two fold. Increasing the ultrasound application time from 15 to 30 minutes, while maintaining the ultrasound intensity at 0.5 w/cm², further increased protein expression (14 and 18 fold respectively), when compared to the control cells.

Trypan blue staining showed that there were differences in the degree of viability depending on the cell type. After ultrasound application at varying intensities (0.25 and 0.5 w/cm²) and time applications (2,5,10,15 and 30 minutes), 80 to 85% of the fibroblasts survived, compared to 70 to 75% of the urothelial cells. The result were confirmed by the MTT assay which was performed after ultrasound application.

### EXAMPLE 2

### Ultrasound Effect on Plasmid DNA Integrity

To ensure that the pGL3-Luc integrity is not affected due to ultrasound exposure (see Example 1), thus affecting the transfection efficiency, two methods were used; agarose gel electrophoresis and cell electroporation. Plates containing pGL3-Luc in medium or PBS (15 mg/mL), were exposed to 1 MHz ultrasound using the same intensities and exposure time as in the in vitro experiment (Example 1, supra). Immediately after exposure, samples of pGL3-Luc DNA were taken and precipitated with 0.4 mL 3M NaOH, 8 mL of 100% EtOH and incubated at −20° C. overnight.

The samples were then spun (11 k RPM, 30 minutes), washed in EtOH and resuspended in 200 μl of Tries-EDTA buffer. The precipitated DNA samples (4 μl) were electrophoresed on 1% agarose gel in the presence of ethidium bromide. Nontreated DNA, i.e., not exposed to ultrasound, and non-precipitated DNA samples were used as controls. The effect of ultrasound on the ability of the pGL3 to express the protein was also evaluated using electroporation techniques. pGL3-Luc DNA samples which were exposed to ultrasound as described, were mixed with fibroblasts cells in a final ratio of 60 mg DNA/10⁶ cells. The cells were then electroporated by application of a potential of 200V for 1 ms, cultured in vitro for three days, harvested and assayed for pGL3-Luc protein expression.

Agarose gel electrophoresis of DNA exposed to 1 MHz ultrasound, showed that the integrity of pGL3-Luc was not affected due to ultrasound application. The migration of DNA exposed to 15 minutes, 20% ultrasound with intensity of 0.25 w/cm² or 30 minutes, was the same as the migration of untreated or untreated-unprecipitated DNA. Increasing the intensity from 0.25 to 0.5 w/cm² (using 15 or 30 minutes of application) did not affect pGL3-Luc integrity. The same phenomena was seen when 50% 0.5 w/cm² was applied for different time application.

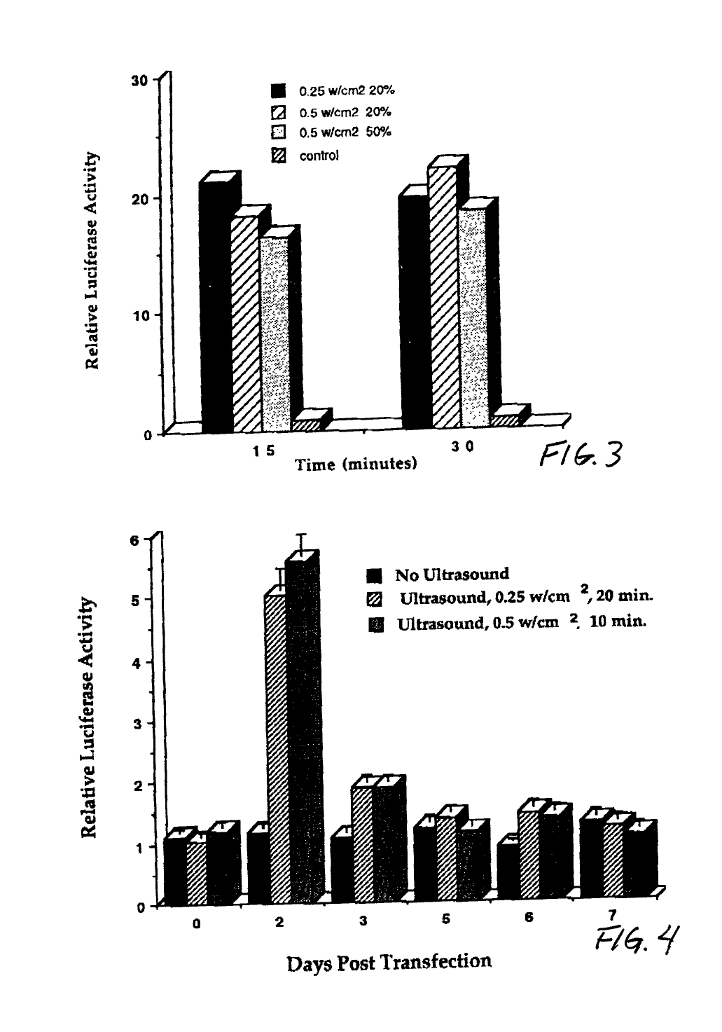

Electroporation has been used for the introduction of protein into cells in vitro. By exposing cells to electrical pulse the cell membrane undergoes a large transmembrane voltage change which causes membrane structure rearrangement, facilitating molecule transportation through the membrane. We used the electroporation technique to determine whether pGL3-Luc affected due to ultrasound treatment was thus affecting transfection efficiency and protein expression level in cells. FIG. 3 shows the level of protein expression in fibroblasts transfected with pGL3-Luc. The DNA was treated with ultrasound, as in the in vitro studies, prior to transfection. The results show clearly that cells are transfected and protein expression is 18-20 fold higher compared to the control cell (i.e., not electroporated). Cells transfected with DNA exposed to different intensities of ultrasound, expressed the same luciferase protein levels. However, these levels were lower than the levels detected in fibroblasts exposed directly to ultrasound and not to electroporation (FIG. 1).

Exposing DNA to 15 or 30 minutes of ultrasound, using intensities of 0.25 or 0.5 w/cm², did not affect pGL3-Luc structure integrity as measured by agarose gel electrophoresis; the migration of the exposed DNA on the gel was the same as the non exposed. The electroporation technique, used for transfection of cells with pGL3-Luc, showed that the cells can be transfected with DNA exposed to ultrasound. However, the transfection efficiency was lower (18-20%) than that obtained when ultrasound was used on fibroblasts (35-37%, FIG. 1). It is known that electroporation, due to the high voltage used, reduces survivability of cells before harvesting and assaying protein expression.

### EXAMPLE 3

### In Vivo Transvesical Delivery of Gene

Female Sprague Dawley rats were anaesthetized by metafane inhalation followed by intramuscular injection of ketamine (45 mg/kg) and xylazine (10 mg/kg). A median laparotomy was performed in all animals in the supine position and the bladder was exposed. Transurethral catheterization was accomplished with a 24 gauge intravenous catheter and the bladder was drained completely. In the experimental group, 0.5 mL of pGL3-Luc (200 mg/mL) was instilled intravesically, and 1 MHz ultrasound (20% duty cycle pulse mode, different intensities and time exposure) was applied directly onto the bladder which was covered with a sterile viscous surgical lubricant. After ultrasound application, the transurethral catheter was removed and the bladder repositioned intrapelvically. The laparotomy was closed in a two layer fashion with interrupted sutures of 3-0 Vicryl and the animals were allowed to recover. The control group received a similar treatment but with no ultrasound application. Rats were sacrificed at different time intervals post treatment and the bladders were harvested, processed and assayed for lucerifase protein expression.

Preliminary data (not shown) showed that pGL3-Luc protein expression in fibroblasts occurs two days post transfection, with electroporation. The cells in the above experiments were harvested 2 days after ultrasound application.

To determine whether ultrasound can affect gene transfection in vivo, rat bladders filled with a solution of pGL3-Luc introduced intravesically were exposed to 1 MHz ultrasound with different intensities and time application. Expression of the pGL3-Luc protein in rat bladders treated for 20 minutes with 20% pulsed ultrasound at an intensity of 0.5 w/cm² was measured. Luciferase protein expression in rats treated with ultrasound was detected two days post treatment. Protein level was five times higher than protein levels detected in the control group which received DNA without ultrasound. No protein expression were detected in rat bladders 3 or 5 days post transfection.

Similar levels of protein expression were detected when ultrasound intensity was elevated to 1 w/cm² while reducing time application to 10 minutes. However, increasing the percent ultrasound transmission from 20 to 50% (i.e., higher energies), while maintaining ultrasound intensity (0.5 w/cm²) for 15 minutes did not affect cell transfection and no protein expression was detected in the bladder.

Gene transfection was also accomplished in vivo. When rat bladders were exposed directly to 1 MHz ultrasound using different intensities and time exposures, protein expression was detected in bladders harvested two days post treatment. Increasing ultrasound power from 0.25 to 1 w/cm², but reducing time application to 10 minutes (i.e., introducing the same energies), gave the same proteins expression levels in bladders. However, higher energies (0.5 w/cm², 50%) did not produce any gene transfection, suggesting that the transfection process requires low energies.

In further experiments, Female Sprague Dawley rats (three for each time point and for each ultrasound parameter) were instilled intravesically with pGL3-Luc and exposed to ultrasound with different intensities and time applications. Luciferase activity was detected two days post treatment in bladders treated with ultrasound (0.25 w/cm² for 20 minutes). The protein levels were five times higher than those detected in the control group which received DNA without ultrasound (FIG. 4). The same levels of protein expression were detected when the ultrasound intensity was increased to 0.5 w/cm² and the time of application was reduced to 10 minutes (FIG. 4). Protein expression was not detected in rat bladders at 3, 5, 6 or 7 days post transfection.

In order to assess, the safety of applying ultrasound energy directly to exposed organs, histological studies were performed. Bladders exposed to different ultrasound intensities and time periods were harvested either before, immediately after, or two days post transfection. There was no evidence of physical damage or inflammatory reaction due to the application of ultrasound. Additional studies were performed on the bladders using the tunnel assay, and these did not show any evidence of apoptosis.

### EXAMPLE 4

### In Vivo Skin Transfection

The skin of nude mice, 4 mice for each time point, were transfected with the plasmid pVEGF-AP using an ultrasound intensity of 2 w/cm² for 20 minutes. This plasmid was used as a preliminary marker for VEGF protein production, which could be easily detected and quantified due to its alkaline phosphatase tag. The production of VEGF-AP protein in the skin was detected on days 2, 3, and 4 post transfection (FIG. 5). No protein production was detected on day 5 post transfection, nor in the control groups in which the DNA was applied topically without ultrasound.

Western blots performed on the skin lysates on days 2 through 5 demonstrated the presence of a 23 kDa protein on days 2, 3 and 4. Human VEGF protein was detected using a monoclonal antibody. The VEGF protein was not detected in untreated skin or in skin harvested from animals which did not receive the DNA or ultrasound.

Similar studies using human pCMV-VEGF which could be localized in the skin immunohistologically have also been performed. VEGF was observed in the epidermal and dermal layers, and the hair follicles on skin sections from days 2 and 3 post transfection.

In FIG. 6 a system 10 for practicing the invention is illustrated including a catheter 12, ultrasound applicator 14, reservoir 16, ultrasound transducer 18, pressure sensor 20, temperature sensor 22 and controller 24. In use the gene formulation is introduced into a taget region (e.g. by ejection or diffusion from reservoir 16 and transducer 18 is activated to induce transfection. The transducer 18 can also receive signals from the target region to monitor the energy. These feedback signals and/or similar monitoring signals from pressure sensor 20 and temperature sensor 22 are relayed (e.g. by electrical leads or telemetry) to controller 24.

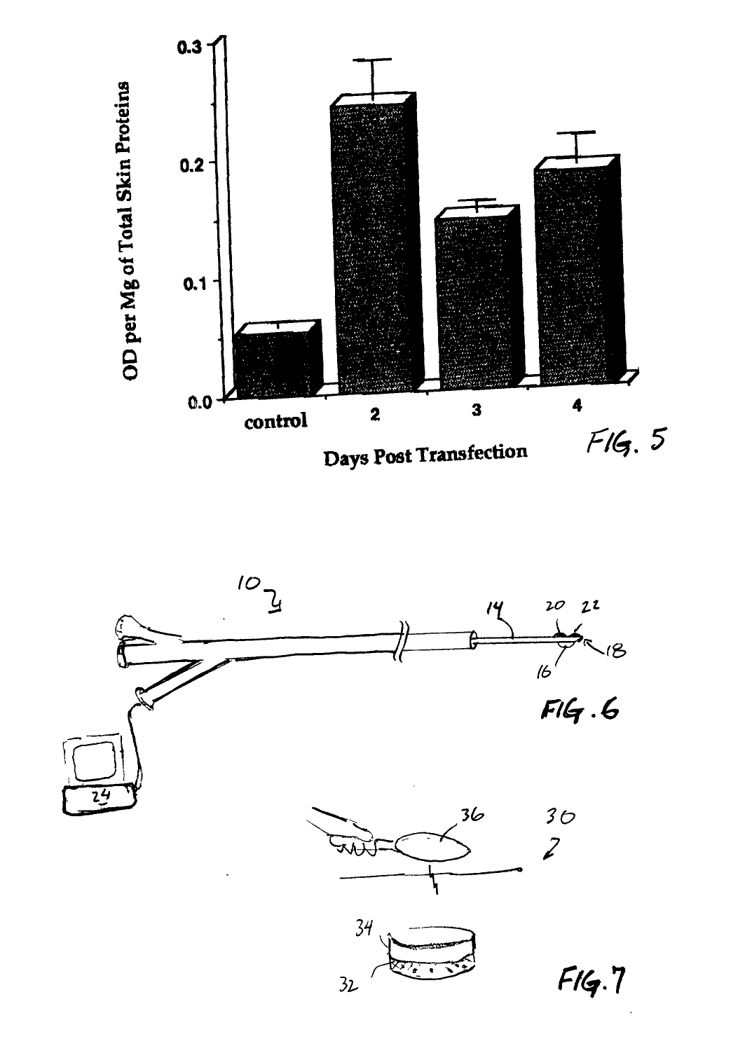

In FIG. 7 an alternative embodiment of the invention is shown in which an implantable system 30 is deployed at a target site. The system includes a matrix 32 carrying the gene formulation and an ultrasound transducer 34. An external RF energy source 36 can be used to power the ultrasound transducer 34.

The contents of all references, patents, and published patent applications cited throughout this application are hereby incorporated by reference.

What is claimed is:

1. A method for promoting cell transfection in a subject, the method comprising:
administering a gene formulation to the subject; and
applying ultrasound energy to the subject, such that cell transfection is promoted in the subject;
wherein the ultrasound energy is provided by an ultrasound source disposed inside the subject's body.

2. The method of claim 1, wherein the source of ultrasound energy is disposed on a catheter.

3. The method of claim 1, wherein the step of administering a gene formulation to the subject comprises administering a gene formulated in liposomes.

4. The method of claim 1, wherein the step of administering a gene formulation to the subject comprises injecting the gene formulation into the subject.

5. The method of claim 1, wherein the step of administering a gene formulation to the subject comprises implanting a degradable matrix incorporating the gene formulation into a subject.

6. The method of claim 1, wherein the step of administering a gene formulation to the subject comprises implanting a degradable matrix incorporating the gene formulation into a subject.

7. The method of claim 6 wherein the step of applying ultrasound further comprises applying ultrasound to degrade the matrix.

8. A system for promoting cell transfection in a subject, the apparatus comprising:
means for administering a gene formulation to the subject; and
an ultrasound applicator for applying ultrasound energy to the subject.

9. The system of claim 8, wherein the means for administering the gene formulation comprises a pharmaceutically acceptable carrier suitable for injection.

10. The system of claim 8, wherein the means for administering the gene formulation comprises a degradable matrix suitable for implantation.

11. The system of claim 8, wherein the means for applying ultrasound energy to the subject is an ultrasound transducer mounted on a catheter.

12. The system of claim 8, wherein the means for applying ultrasound energy is a needle operatively connected to an ultrasound transducer.

13. The system of claim 8, wherein the means for applying ultrasound energy is an implantable ultrasound transducer.

14. The system of claim 8, wherein the gene formulation further comprises at least one plasmid.

15. The system of claim 8, wherein the gene formulation further comprises a lipsome delivery vessel.

16. A catheter for promoting cell transfection in a subject, the catheter comprising:
an elongate tubular assembly having at least one lumen and an opening at or near the distal end of the catheter; the distal end of the lumen being in fluid communication with the opening; and
a source of ultrasound energy disposed at or near the distal end of the catheter.

17. The catheter of claim 16, wherein the proximal end of the catheter is provided with an adapter for introduction of a fluid into the lumen.

18. The catheter of claim 17, wherein the catheter further comprises a fluid reservoir fluidly communicating with to the adapter.

19. The catheter of claim 16, wherein the catheter further comprises an ultrasound receiver capable of monitoring the level of ultrasound energy applied to a target site.

20. The catheter of claim 19, wherein the catheter further comprises a controller for receiving signals from the ultrasound receiver and for controlling the level of ultrasound energy applied to a target site in accordance with the received signals.
